(19) United States
(12) Patent Application Publication
(10) Pub. No.: US 2011/0280952 A1
(43) Pub. Date: Nov. 17, 2011

(54) Platelet Lysate and Bioadhesive Compositions Thereof for the Treatment of Mucositis

(75) Inventors: Carla Marcella Caramella, Pavia (IT); Maria Cristina Bonferoni, Pavia (IT); Silvia Stefania Rossi, Pavia (IT); Giuseppina Sandri, Pavia (IT); Franca Ferrari, Pavia (IT); Cesare Giuseppe Perotti, Pavia (IT); Claudia Del Fante, Pavia (IT)

(73) Assignees: BIOMED DEVICE S.R.L., Reggello (FI) (IT); UNIVERSITA' DEGLI STUDI DI PAVIA, Pavia (IT)

(21) Appl. No.: 13/149,311

(22) Filed: May 31, 2011

Related U.S. Application Data
(63) Continuation of application No. PCT/IT2008/000744, filed on Dec. 5, 2008.

(51) Int. Cl. A61K 35/14; A61P 29/00; A61P 27/02; A61K 35/16

(52) U.S. Cl. 424/530; 424/532

(57) ABSTRACT

The present invention concerns the use of platelet lysate for treating and/or preventing mucositis. Moreover, a mucoadhesive composition comprising such a platelet lysate for the therapy and/or prevention of mucositis and of corneal lesions is described.

This application is a continuation of international application PCT/IT2008/000744 filed Dec. 5, 2008.

### BACKGROUND OF THE INVENTION

The present invention finds application in the field of medicine; more specifically, it concerns compositions for the prevention and treatment of mucositis and of corneal lesions.

In many studies, both in vitro and in vivo, it has been demonstrated how effective growth factors are in promoting cellular proliferation and differentiation, chemiotaxis and synthesis of the extracellular matrix involved in the tissue healing process. Such factors have also been delivered in different release systems. For example, porous three-dimensional polymeric matrices based upon copolymers of lactide and glycolide (PLG) have been proposed to deliver angiogenic factors like the endothelial vascular growth factor (M. H. Sheridan et al., Bioabsorbable polymer scaffolds for tissue engineering capable of sustained growth factor delivery, J. Controlled Release 64, 91-102 (2000)). Gelatine-based biodegradable hydrogels have been studied for the release of the transforming growth factor (TGF-β1) in order to promote bone regeneration (M. Yamamoto et al., Bone regeneration by transforming growth factor b1 released from a biodegradable hydrogel, journal of Controlled Release 64, 133-142 (2000)). Other authors have studied the interaction of the basic fibroblast growth factor (bFGF) with biodegradable hydrogels of gelatine (Y. Tabata et al., In vitro sorption and desorption of basic fibroblast growth factor from biodegradable hydrogels, Biomaterials 19, 1781-1789 (1998)).

Often, the success of the therapy greatly depends upon the location and the anatomical-physiological characteristics of the site covered by the lesion, which, for example, can be difficult to reach or subject to removal mechanisms of the therapeutic agent.

This problem occurs, for example, in the case of mucositis, for which no entirely satisfactory therapeutic agent has yet been described.

Mucositis are lesions that can occur following therapeutic treatments like, for example, radio- and chemo-therapy in cancer treatment. Such mucositis can greatly influence the quality of life with symptoms often so serious as to require, in the case of anti-tumour therapy, that the therapy be halted or shortened or that the drug doses be reduced. It may also become necessary to have parenteral analgesia, hospitalisation and, in the case of buccal mucositis, parenteral nutrition.

The compounds for therapy used up to now have proven to have little activity, are difficult to apply or else, once applied, easily move away or are removed from the site, for example, by physiological secretions or by swallowing movements in the case of buccal mucositis.

Consequently, the active ingredients stay in contact with the mucosa to be treated for an insufficient time, thus slowing down the healing process.

Indeed, the therapeutic effect is not carried out apart from to a minimal extent and, because the first symptoms of healing can occur, it is necessary to perform a continuous and repeated application of the therapeutic agent, which is not always possible, is inconvenient and often unacceptable to the patient.

Similar problems are also encountered in the case of corneal lesions; indeed, the site involved is difficult to reach and the residency time of the active ingredient, often cannot be ensured for a long time period due to lachrymal secretions.

Therefore, therapeutic compositions are necessary comprising an agent or a pool of effective therapeutic agents and that are delivered equally effectively to treat mucositis and/or corneal lesions.

### SUMMARY OF THE INVENTION

In a first aspect, the present invention concerns the use of platelet lysate as a medicament and, more specifically, as medicament for the therapy and/or prophylaxis of mucositis, according to claim 1.

The lysate preferably has the characteristics detailed in claims 2 and 3.

The present invention comprises, in addition, a pharmaceutical preparation containing the platelet lysate of the invention according to claims 6 and 7.

In a further aspect, the present invention concerns a mucoadhesive composition comprising a carrier and a platelet lysate, according to claim 8 and having the further preferable characteristics of dependent claims 9 to 19 and 36.

In a further object, the mucoadhesive compositions of the invention are described as a medicament, according to claim 20.

In a preferred aspect, the compositions are described as a medicament for treating mucositis and corneal lesions, according to claim 21 and dependent claims 22 to 26.

In a further aspect of the invention, a process for preparing the mucoadhesive compositions of the invention is described, according to claims 27 to 35.

In an additional aspect, the composition of the invention comprises pharmaceutically active agents according to claim 36.

A further aspect of the invention is a method for the therapy and/or prophylaxis of mucositis according to claims 4 and 5.

Yet another aspect of the invention concerns a method for prophylaxis and/or therapy of mucositis and corneal lesions according to claims 37 and 38.

### BRIEF DESCRIPTION OF THE DRAWINGS

FIG. 1 shows the viscosity profiles observed for the carrier 1 at time 0 and after one month of storage at 4-8° C. (average values±SD; n=3).

FIG. 2 shows the values of the “maximum adhesion force” parameter evaluated for just the carrier and for the composition 1 (lysate:carrier weight ratio 1:1, see Example 1) (average values±SD; n=9)

FIG. 3 shows the values of the “maximum adhesion force” parameter evaluated for just the carrier and for the composition 2 (lysate:carrier weight ratio 1:1, see Example 2) (average values±SD; n=9)

FIG. 4 shows the cellular proliferation values determined at different times (time zero T0, 2 days T2, 7 days T7, 10 days T10 and 15 days T15) on fibroblasts for just lysate and for the composition 1 (lysate:carrier weight ratio 1:1, see Example 1). The concentration of lysate in the composition is the same as is used for just lysate.

By control we mean the % proliferation of viable cells obtained in the absence of platelet lysate (LP) and of composition.

LP 1:20 and LP 1:40: % proliferation of viable cells obtained in the presence of platelet lysate (LP) at two different dilutions, 1:20 and 1:40, with culture medium lacking cellular growth factors.

Composition 1:20 and composition 1:40: % proliferation of viable cells obtained in the presence of platelet lysate (LP) carried in the composition at two different dilutions, 1:20 and 1:40, with culture medium lacking cellular growth factors.

FIG. 5 shows the cellular proliferation values determined at different times (time zero T0, 7 days T7, 10 days T10, 15 days T15 and 20 days T20) on a rabbit's corneal epithelial cellular lines for just lysate and for composition 2 (lysate:carrier weight ratio 1:1, see Example 2). The concentration of the lysate in the composition is the same as is used for just lysate.

By control we mean the % proliferation of viable cells obtained in the absence of platelet lysate (LP) and of composition.

LP 1:20 and LP 1:40: % proliferation of viable cells obtained in the presence of platelet lysate (LP) at two different dilutions, 1:20 and 1:40, with culture medium lacking cellular growth factors.

Composition 1:20 and composition 1:40: % proliferation of viable cells obtained in the presence of platelet lysate (LP) carried in the composition at two different dilutions, 1:20 and 1:40, with culture medium lacking cellular growth factors.

### DETAILED DESCRIPTION OF THE INVENTION

In a first object, the present invention concerns the use of platelet lysate for the therapy and/or prophylaxis of mucositis.

In the present invention, said platelet lysate, abbreviated LP, can be “autologous”, i.e. obtained from platelet-rich plasma (PRP) of the same patient for whom the composition of the invention is intended, or else “allogenic”, i.e. formed from platelet-rich plasma (PRP) obtained from a subject known as a donor.

The use in therapy of autologous LP has the advantage of eliminating the risks linked to the transmission of disease. Moreover, the use of allogenic LP has the advantage of providing a large quantity of material having standard characteristics in terms of type and quantity of growth factors contained in it. To obtain autologous lysate, a peripheral venous blood sample suitably anti-coagulated, for example with acid citrate dextrose, is taken from the patient and subjected to centrifuging at 1000 rpm to separate the platelet-rich plasma (PRP) from the other corpuscular elements of blood like red or white blood cells. Then follows the rapid freezing of the PRP to a temperature of −80° C. for 10 minutes and unfreezing of the sample without use of any exogenous substance, so as to cause the platelet membrane to break, determining the release by lysis into the plasma of the growth factors contained in the alpha granules of the platelets. On the other hand, in the case in which a sample of PRP collected through productive apheresis performed on donors through a cellular separator and then divided into aliquots of the volume of about 50 ml of platelets is used, it can be directly frozen and then unfrozen, according to the same methodologies described above to obtain autologous lysate, in this way preparing allogenic platelet lysate.

In both cases, however, the lysate obtained is rich in numerous growth factors.

Such a lysate has proven to surprisingly and unexpectedly possess much greater therapeutic properties, in promoting tissue repair both at mesenchymal and epithelial level, compared to known preparations comprising isolated growth factors.

Once prepared, the lysate is characterised through immunological dosage (ELISA) to evaluate the concentration of Human PDGF-AB: a particularly representative growth factor belonging to the family of human platelet growth factors.

The platelet lysate is then lyophilised to ensure its biological-therapeutic activity over time after having added suitable lyostabilizers, like, for example, trehalose, mannitol or glycine.

Such a platelet lysate can be applied to the site affected by mucositis, like, for example, the buccal, gastro-intestinal, nasal, vaginal or rectal mucosa or by vesical instillation.

In particular, such application can occur in the form of solution with possible addition of viscosifier agents like for example rubbers or aluminosilicates, sweeteners and/or flavourings. In terms of dosage, the application of the platelet lysate can occur in one of the forms indicated above and for an amount of between about 50-100 mg of lysate per cm² of mucosa.

In a further object, the present invention concerns a bioadhesive composition comprising a carrier and a platelet lysate.

By bioadhesive composition, we mean a composition capable of adhering to a physiological, human or animal surface, for example a tissue or a mucosa.

In the present invention, by “carrier” it is meant an aqueous solution of one or more mucoadhesive agents.

In particular, for the purposes of the present invention, by mucoadhesive agent we mean a compound or a mixture of compounds able to adhere to the mucosa, i.e. to the coating of the body cavities in communication with the outside through natural orifices. Such mucoadhesive agents, in particular, can be selected from polyacrylic acid (carbopol 974-P NF) or cross-linked polyacrylic acid like, for example, carbomer or polycarbophyl, or other derivatives of polyacrylic acids like, for example, polymethylmethacrylates; cellulose or derivatives of cellulose, like, for example, sodium carboxymethylcellulose, hydroxypropylcellulose, hydroxypropylmethylcellulose; other polysaccharides, like, for example, chitosan and its pharmaceutically acceptable salts, like, for example, chitosan glutamate, or, furthermore, polysaccharides like alginates, pregelatinised starches, pectins; glycosoaminoglycans, like, for example, hyaluronic acid, chondroitin sulphate; polyvinylpyrrolidone; gelatine; or mixtures of the mucoadhesive agents listed above.

The carrier is thus obtained by dissolution of one of the mucoadhesive agents indicated above in water, for example, distilled water or water in physiological solution, i.e. with 0.9% NaCl weight/volume, through agitation and subsequent addition of sweeteners and flavourings. Moreover, neutralising and/or buffer agents can be added, so that the pH is compatible with the physiological environment of the site of application.

For example, in the case in which the composition of the invention foresees buccal application, the pH values of the composition must be between 5.5 and 7.5 and, preferably between 6 and 7, even more preferably a pH value of about 6.5.

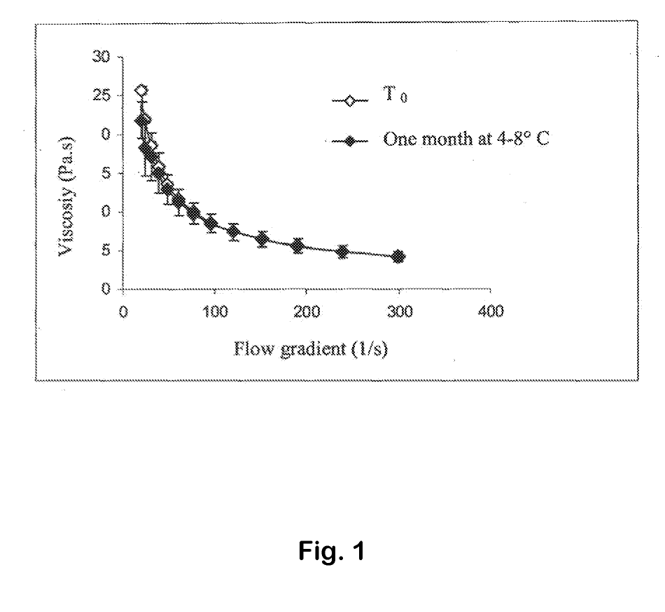

The carrier thus obtained can be conserved, by sterilisation or addition of preservatives known in the field, for up to 15-30 days at a temperature of 4-8° C. maintaining its rheological characteristics, like viscosity, as illustrated in FIG. 1 for the carrier relative to Example 1.

In particular, FIG. 1 shows, as a function of the flow gradient, taken to be the ratio between the difference between the maximum and minimum flow speed in the thickness of the fluid and the thickness of the fluid itself and expressed in 1/s (P. Colombo et al, Principi di Tecnologie Farmaceutiche, Casa Editrice Ambrosiana, Milano, 2004), the viscosity, expressed in Pascals (Pa), for the carrier made according to the present invention, at time zero, i.e. just prepared, and one month after its preparation.

Before being mixed with the other components of the composition of the invention, the carrier, prepared as described above, is sterilised in an autoclave, according to methodologies known to the man skilled in the art.

The mucoadhesive composition of the invention comprises, as stated above, in addition to the carrier containing the mucoadhesive agent, a platelet lysate (LP).

Said lysate, in particular, is the lysate obtained according to what has been described above and is mixed with the “carrier” obtained as detailed above.

The mixing of the carrier with the platelet lysate, in particular, is carried out so as to obtain a homogeneous composition.

More specifically, the composition of the invention comprises platelet lysate and the carrier, in a weight ratio of between about 1:6 and 6:1. In a preferred aspect, such a weight ratio is between about 1:2 and 2:1 and, even more preferably, it is about 1:1.

The composition of the invention has rheological properties, like viscosity, which allow easy application to the mucosae, but, at the same time, a surprising and unexpected mechanical strength.

In this way, the composition has demonstrated that it remains for an advantageously long time at the site on which it has to carry out the therapeutic and/or prophylactic activity.

Consequently, the number of applications on the site affected by mucositis or corneal lesion can be advantageously reduced, whilst still ensuring effectiveness and speed in acting therapeutically.

For example, in the case in which the composition of the invention foresees a buccal application, the pH values of the composition must, as mentioned above, be between 5.5 and 7.5, preferably between 6 and 7, a pH value of about 6.5 being even more preferable (see Table I shown below).

The respective viscosity values measured at flow gradient equal to 50 s⁻¹ (see, in particular, P. Colombo et al, Principi di Tecnologie Farmaceutiche, Casa Editrice Ambrosiana, Milano, 2004) of said compositions are between 1 and 8 Pa·s, preferably between 1.5 and 6 Pa·s and even more preferably they are between 2 and 4.5 Pa·s (see Table I below).

TABLE 1

| pH | Apparent viscosity (Pa · s) |
|---|---|
| 5.5-7.5 | 1-8 |
| 6-7 | 1.5-6 |
| 6.5 | 2-4.5 |

One of the advantages of the composition of the invention is represented by its properties of mucoadhesion, i.e. the ability to interact with the mucus and/or the mucosa of application so as to remain for an extended time in the site of application.

Said properties are measured by evaluating the detachment force, i.e. the force necessary to detach a layer of the composition by a mucin gel. The test foresees using a commercial apparatus (TA XTPlus) or another analogous apparatus for measuring the traction force. In brief, in the adopted test, widely described in literature, a layer of composition is positioned on a surface of inert material and arranged in contact with a commercial mucin solution mimicking the surface of the mucosa (the biological substrate). Then the force necessary to detach the composition from the biological substrate is measured (see, for a general reference, S. Rossi et al., Chitosan ascorbate: a chitosan salt with improved penetration enhancement properties. Pharm. Dev. Technol. 13 (6), (2008), 513-21).

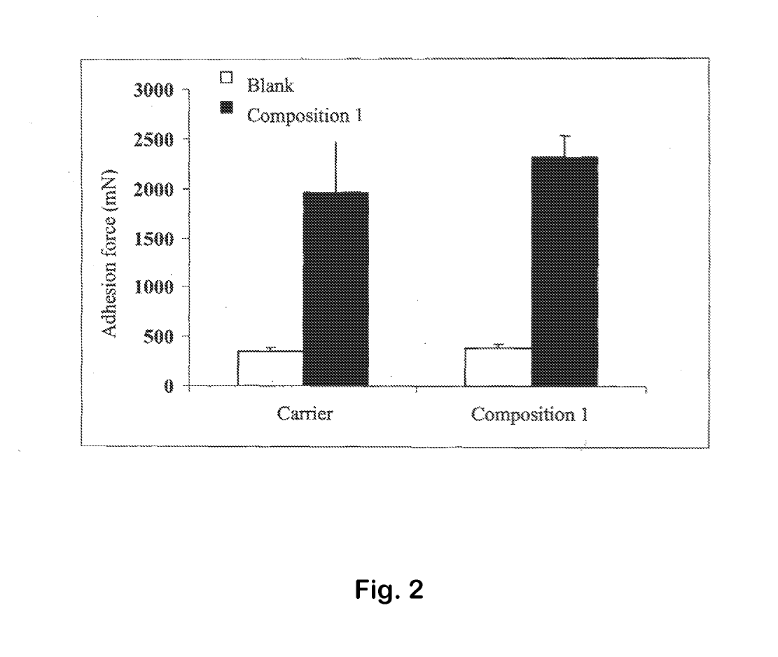

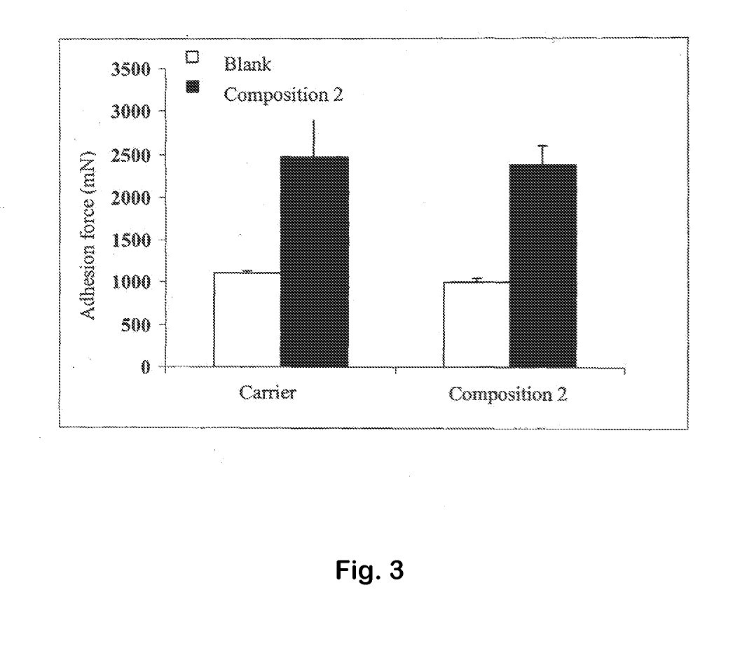

The data relative to the compositions of Examples 1 and 2 are illustrated in FIGS. 2 and 3.

In particular, by “maximum adhesion force” we mean the maximum force recorded during the detachment experiment of the composition from the biological substrate.

From FIGS. 2 and 3, it can clearly be seen that both the carrier and the compositions comprising such a carrier are characterised by greater detachment force values in the presence of mucin compared to the blank, i.e. to measurements carried out on the composition using buffer in the test instead of the biological substrate.

The experimental data, therefore, demonstrates the advantageous and unexpected ability of the compositions to adhere to the biological substrate.

Indeed, the lack of significant differences between the maximum force values observed for just the carrier with respect to the composition of the invention indicates that the mixing of the carrier with the platelet lysate (LP) surprisingly does not affect the ability of the mucoadhesive agent to carry out its own activity.

Moreover, the compositions of the invention unexpectedly demonstrate a further advantage, since they are able to release the growth factors present in the platelet lysate without the action promoting cellular proliferation being compromised.

The compositions of the present invention, in particular, can be prepared in the form of a gel, which is a semi-solid system, as described in Examples 1 and 2; as a film, obtained by drying from a gel from; or as a “sponge like” system, obtained by lyophilization of a gel form. Both the film form and the “sponge like” form are intended for direct local application on the lesions. Alternatively, the composition can also be dried in the form of particulate or micro-particulate dispersions, for example by nebulisation, representing a powder to be applied directly or after pre-suspension in a suitable medium.

Such dispersions, in particular, increase the contact surface between therapeutic agent and mucosa to be treated.

In particular, the film, “sponge-like”, and micro-particulate and particulate forms conserve the adhesive properties of the gel from which they are obtained.

Films, sponge like systems and particulate or micro-particulate systems can be obtained according to methods commonly known to men skilled in the art (see, for a general reference, P. Colombo et al, Principi di Tecnologie Farmaceutiche, Casa Editrice Ambrosiana, Milano, 2004)

In addition, the mucoadhesive composition of the present invention can be prepared, for example in a hospital facility, shortly before application to the patient, for example by rehydration of the composition of the invention prepared in lyophilized form with a suitable agent.

The mucoadhesive composition according to the invention can also comprise pharmaceutically acceptable technological agents like emollients, sweeteners, like, for example, saccharin, flavourings, like mint, strawberry or liquorice flavour, pH modifiers, like NaOH solutions or buffers, preservatives or viscosifiers, as known to the skilled in the art.

With regard to the therapeutic doses of the compositions of the invention, they can be applied, for example, in quantities of between about 100-200 mg comprising platelet lysate and carrier in a 1:1 weight ratio per cm² of mucosa.

According to a further aspect, the present invention concerns the use of the compositions of the invention comprising a carrier and a platelet lysate as a medicament.

More specifically, said compositions can be applied onto mucosae for the prophylaxis and/or therapy of mucositis or else for the prophylaxis and/or therapy of corneal lesions.

Mucositis is an inflammation that affects the mucosae and, as described above, it can have a different etiogenesis, including anti-tumour radio- and chemotherapy.

The seriousness of mucositis can be evaluated in grades, from I to IV, according to the extension and the symptoms, as follows:

| Mucositis grade | Symptoms |
|---|---|
| I | Erithema of the mucosa |
| II | Patchy or pseudomembranous ulceration |
| III | confluent or pseudo-membranous ulcerations; bleeding |
| | with small trauma |
| IV | Necrosis of the tissue with significant spontaneous |
| | bleeding; consequences placing life at risk |

Corneal lesions, on the other hand, can be caused by foreign or intraocular bodies and are particularly harmful if not recognised and not cured. Moreover, if no therapeutic intervention is made, there can easily be secondary infections.

In these cases, the application of the compositions of the invention can take place on mucositis of the buccal, gastro-intestinal, nasal, vaginal and rectal mucosa, by vesical instillation, as well as, in the case of corneal lesions, on conjunctival mucosa and on the corneal surface.

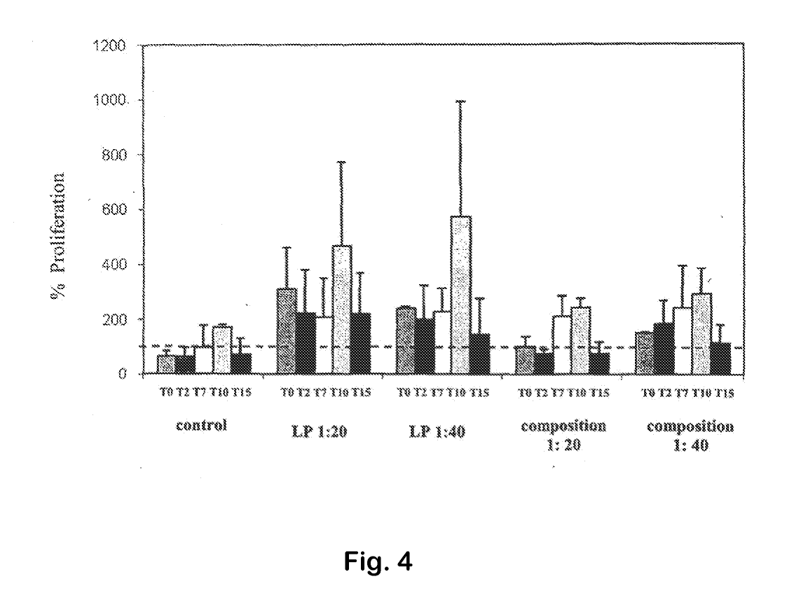

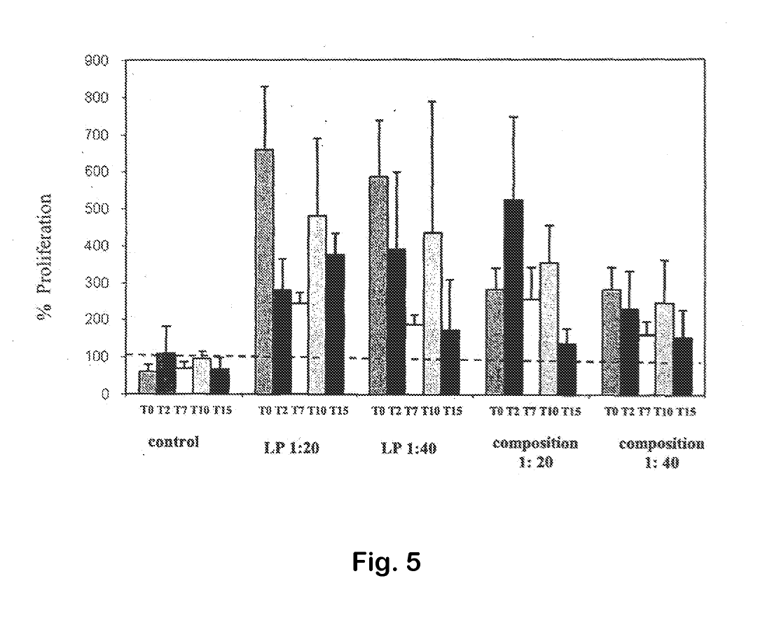

The therapeutic properties of the compositions of the invention can be checked “in-vitro” through cellular proliferation measurements made on different cellular lines; in the present case a rabbit's fibroblasts and corneal epithelial lines have been used, as illustrated in FIGS. 4-5.

In particular, said cellular proliferation measurements are made by placing the suitably diluted composition in contact for 24 hours with the cells grown in wells. Then the number of live cells is evaluated through a cell survival test (neutral red (NR) assay (Tox Kit 4, Sigma-Aldrich, Milano, Italy) (for a general reference, see, for example, M. C. Bonferoni et al., Chitosan citrate as multifunctional polymer for vaginal delivery. Evaluation of penetration enhancement and peptidase inhibition properties. Eur. J. Pharm. Sci. 33, (2008) 166-176).

The present invention is described further hereafter with examples given purely for illustrative purposes of the different aspects of the making of the compositions object of the invention and they are not intended to limit the invention in any way.

### Example 1

### Preparation of composition 1

a) Preparation of the Carrier

A gel based upon polyacrylic acid (carbopol 974-P NF) is prepared having the following composition:
carbopol 5% (p/p)
saccharin 0.2% (p/p)
flavourings 0.2% (p/p)
NaOH4N up to pH 7.

In particular, the saccharin is dissolved in a physiological solution (0.9% weight/volume of NaCl) through magnetic agitation. Carbopol is added and the mixture is kept under agitation until complete hydration (about 12 hours). When hydration is complete, a flavouring is added, for example mint, liquorice or strawberry. The formulation is then buffered to pH 7 through the use of a solution of NaOH4N in order to obtain the gelification of the carbopol and thus a formulation having a pH compatible with the mucosa of the oral cavity.

The carrier thus prepared is then poured into a heat-resistant glass bottle, the empty space at the top is filled with nitrogen and it is subjected to sterilisation with damp heat in an autoclave for 15 minutes at a temperature of 121° C.,

b) Preparation of Composition 1

The carrier obtained in step a) is then mixed with platelet lysate in a 1:1 weight ratio obtaining a final carbopol concentration of about 2.5% by weight of the total of the composition.

### Example 2

### Preparation of Composition 2

a) Preparation of the Carrier for Composition 2

A gel based upon chitosan and hydroxypropylmethylcellulose (HPMC) was prepared with the following composition:
chitosan glutamate 6% (p/p)
HPMC 2% (p/p)

The hydroxypropylmethylcellulose is hydrated with a minimum amount of distilled water.

Chitosan glutamate is added until a concentration of 6% p/p is obtained and the whole thing is brought up to volume with water and kept under mild agitation until the complete hydration of the polymers.

The pH of the carrier thus prepared is equal to 5.5.

The gel is then poured into a heat-resistant glass bottle, the empty space at the top is filled with nitrogen and it is subjected to sterilisation with damp heat in an autoclave for 15 minutes at 121° C.

b) Preparation of Composition 2

The carrier obtained in step a) is then mixed with platelet lysate in a 1:1 weight ratio up to a final concentration of chitosan glutamate of 3% (p/p) and of HPMC of 1% (p/p).

### Example 3

### Characteristics of Composition 1

The composition 1 has a pH value equal to about 7 and rheological properties suitable for application in the oral cavity: apparent viscosity at 50 s⁻¹ equal to about 4.5 Pa·s.

The composition has properties of mucoadhesion as illustrated in FIG. 2.

The composition maintains the ability to promote cellular proliferation even after conservation at a temperature of 4-8° C. for 10-15 days as represented in FIG. 4.

The composition 1 in a preliminary in vivo study proved effective in the treatment of mucositis in five patients suffering from mucositis of the oral cavity of grade III-IV.

In particular, in 4 such patients the response involved a recovery of the integrity of the damaged of between 50% and 100% and only in one case was it less than 50%. In just one patient with grade I mucositis was there no response.

No patients suffered local infections as a result of application of the lysate in mucoadhesive carrier.

### Example 4

### Characteristics of Composition 2

Composition 2 has rheological properties suitable or application in the oral cavity: apparent viscosity 50 s⁻¹ equal to about 3 Pa·s.

The composition has properties of mucoadhesion as illustrated in FIG. 3.

The composition has demonstrated that it maintains the ability to promote cellular proliferation even after storage at a temperature of 4-8° C. for 10-15 days as represented in FIG. 5.

We claim:

1. A mucoadhesive composition comprising a platelet lysate and a carrier selected from the group comprising: polyacrylic acid and its derivatives; glucosaminoglycans and its derivatives; polyvinylpyrrolidone; gelatine; chitosan and its salts; alginates; pregelatinised starches; pectins; cellulose and its derivatives; and their mixtures.

2. The mucoadhesive composition according to claim 1, wherein said derivatives of polyacrylic acid comprise carbomer, polycarbophyl and polydimethylmethacrylates.

3. The mucoadhesive composition according to claim 1, wherein said glucosaminoglycans comprise chondroitin sulphate and hyaluronic acid.

4. The mucoadhesive composition according to claim 1, wherein said derivatives of cellulose comprise sodium carboxymethylcellulose, hydroxycellulose and hydroxypropylmethylcellulose.

5. The mucoadhesive composition according to claim 1, wherein said platelet lysate is obtainable from platelet-rich plasma.

6. The mucoadhesive composition according to one of claim 1, wherein said carrier and said platelet lysate are contained in a weight ratio comprised between about 6:1 and 1:6.

7. The mucoadhesive composition according to claim 6, wherein said carrier and said platelet lysate are contained in a weight ratio comprised between about 1:2 and 2:1.

8. The mucoadhesive composition according to claim 1, in the form of a semisolid system, of film obtained by drying a semisolid system, of a lyophilized system, of particle or micro-particle dispersions.

9. The composition according to claim 8, wherein said lyophilized system is of the “sponge-like” type.

10. A method for the prophylaxis and/or therapy of mucositis or of corneal lesions, comprising the step of applying to the mucosae of a patient in need thereof of a therapeutically amount of the mucoadhesive composition of claim 1.

11. The method for the prophylaxis and/or therapy of mucositis or of corneal lesions according to claim 10, wherein said amount is comprised between about 50 mg and 100 mg of platelet lysate per cm² of mucosae.

12. The method for the prophylaxis and/or therapy of mucositis or of corneal lesions according to claim 10, wherein said mucoadhesive composition is applied by buccal, gastro-intestinal, nasal, vaginal, rectal application, by vesicle instillation or by application on the conjunctival mucosa or on the corneal surface, respectively.

13. The method for the prophylaxis and/or therapy of mucositis or of corneal lesions according to claim 10, wherein said composition has a pH of between about 5.5 and 7.5 and a viscosity of between about 1 and 8 Pa·s.

14. The method for the prophylaxis and/or therapy of mucositis or of corneal lesions according to claim 13, wherein said composition has a pH comprised between about 6 and 7 and viscosity of between about 1.5 and 6 Pa·s.

15. The method for the prophylaxis and/or therapy of mucositis or of corneal lesions according to claim 13, wherein said composition has a pH of about 6.5 and viscosity comprised between about 2 and 4.5 Pa·s.
